(19) United States
(12) Patent Application Publication
(10) Pub. No.: US 2021/0139841 A1
(43) Pub. Date: May 13, 2021

(54) COMPOSITION FOR DEGRADATION OF AFLATOXIN COMPRISING ASPERGILLUS CULTURE FILTRATE AS EFFECTIVE COMPONENT AND USES THEREOF

(71) Applicant: SKYANGELBIO Corp., Seoul (KR)

(72) Inventors: Jaehyuk YU, Madison, WI (US); Ahmad Fakhri Mohammad ALSHANNAQ, Madison, WI (US); Dasol CHOI, Madison, WI (US)

(21) Appl. No.: 16/681,004

(22) Filed: Nov. 12, 2019

(51) Int. Cl. C12N 1/14; A23L 3/3571; A23L 33/135; A23K 30/00; A23K 10/18

(52) U.S. Cl. CPC C12N 1/14; A23L 3/3571; A23V 2002/00; A23K 30/00; A23K 10/18; A23L 33/135

(57) ABSTRACT

A composition for degradation of aflatoxin includes Aspergillus culture filtrate as an effective component and uses thereof, and it is expected that, in the field of food products and animal feeds for which biodegradation of fungal toxin (in particular, aflatoxin) is required, the composition can be advantageously used as a novel material that can maintain the activity of degrading fungal toxin even at high temperatures.

### TECHNICAL FIELD

The present invention relates to a composition for degradation of aflatoxin comprising Aspergillus culture filtrate as an effective component and uses thereof.

### BACKGROUND ART

Aflatoxins are a group of highly toxic secondary metabolites produced by koji mold mushrooms. As the most potent foodborne carcinogen, aflatoxins threaten global food safety by contaminating ˜25% of the world food supply and also threaten the public health. Since the discovery of aflatoxins in early 1960s to the present time, several approaches to control and remove aflatoxins have been developed, and some are used in fields. However, no technologies or related products are considered fully effective for removing aflatoxins, and, currently, none can be directly used in food. Aflatoxins are maintained in highly stable state during food processing such as boiling or even autoclaving. In the absence of effective measures and with climate changes, aflatoxin problems are predicted to increase continuously.

Meanwhile, in Korean Patent Application Publication No. 2015-0007918, “Aflatoxin production inhibitor and method for controlling aflatoxin contamination” is described, and, in Korean Patent Registration No. 0380535, “Method for controlling production of aflatoxin using antagonistic microorganism CP220 and fermented bean food product and animal feed using it” is described. However, the composition for degradation of aflatoxin of the present invention which comprises Aspergillus culture filtrate as an effective component and uses thereof are not disclosed before.

### DETAILED DESCRIPTION OF THE INVENTION

### Technical Problems to be Solved

The present invention is devised under the circumstances described above, and the inventors of the present invention found that, during the course of carrying out a study for developing a method for effective degradation of aflatoxin as threats to global public health safety, the culture filtrate of specific Aspergillus species exhibits an excellent aflatoxin-degrading activity. In particular, it was found that the Aspergillus culture filtrate maintains the aflatoxin-degrading activity not only under room temperature conditions but also under heating conditions like 100° C. By analyzing and establishing the composition for optimizing the aflatoxin-degrading activity while minimizing the use amount of constitutional materials in culture medium for food product that can be safely taken by human, the inventors completed the present invention.

### Technical Means for Solving the Problems

To solve the problems that are described in the above, the present invention provides a composition for degradation of fungal toxin comprising Aspergillus culture filtrate as an effective component.

The present invention further provides a method for degradation of fungal toxin including contacting a subject for degradation with the aforementioned composition.

The present invention further provides a method for production of Aspergillus culture filtrate having an activity of degrading fungal toxin including steps of inoculating Aspergillus conidia to a culture medium followed by culturing; and filtering a culture liquid of the Aspergillus, and it also provides an Aspergillus culture filtrate having an activity of degrading fungal toxin that is produced by the aforementioned method.

The present invention further provides a food additive comprising an Aspergillus culture filtrate having an activity of degrading fungal toxin.

The present invention still further provides an animal feed additive comprising an Aspergillus culture filtrate having an activity of degrading fungal toxin.

### Advantageous Effect of the Invention

Compared to techniques of a related art, the composition for degradation of fungal toxin according to the present invention can degrade aflatoxin with higher efficiency, and, as the activity of degrading fungal toxin is maintained in very stable state even under heating conditions like 100° C., it is expected that the composition of the present invention can be advantageously used for processings like treatment at high temperatures. Accordingly, it is expected that, in the field of food product and animal feed for which biodegradation of fungal toxin (in particular, aflatoxin) is required, the composition of the present invention can be advantageously used as a novel material that can maintain the activity of degrading fungal toxin even at high temperatures.

### BRIEF DESCRIPTION OF THE DRAWINGS

FIG. 1 is a flow chart showing the process of producing D-Tox.

FIG. 2 shows a protocol for degradation of aflatoxin (AFB1).

FIG. 3 and FIG. 4 show the result of analyzing aflatoxin-degrading activity of D-Tox depending on reaction temperature and time, in which FIG. 3 shows the result of determining the aflatoxin-degrading activity of D-Tox at different time points (aflatoxin was 5,000 ppb) and FIG. 4 shows the result of determining the aflatoxin-degrading activity of D-Tox on different number of days (aflatoxin was 1,500 ppb).

FIG. 5 shows the result of aflatoxin-degrading activity of D-Tox under high temperature conditions (100° C.).

FIG. 6 shows the result of comparing the aflatoxin-degrading activity of various strains of Aspergillus oryzae and other strains belonging to genus Aspergillus, in which, after the reaction with 10 ppm aflatoxin for 24 hours at 30° C., the reduction amount of aflatoxin was calculated compared to the chromatogram area at initial concentration.

FIG. 7 shows the result of analyzing the aflatoxin-degrading activity of D-Tox depending on various pH conditions.

### DETAILED DESCRIPTIONS OF THE INVENTION

To achieve the object of the present invention, the present invention provides a composition for degradation of fungal toxin comprising Aspergillus culture filtrate as an effective component.

With regard to the composition for degradation of fungal toxin of the present invention, the fungal toxin can be preferably aflatoxin, but not limited thereto.

Furthermore, Aspergillus can be Aspergillus oryzae (A. oryzae), Aspergillus terreus (A. terreus), Aspergillus sojae (A. sojae), Aspergillus nidulans (A. nidulans), Aspergillus fumigatus (A. fumigatus), or Aspergillus flavus (A. flavus), but not limited thereto. The culture filtrate originating from those strains is characterized by its remarkably excellent aflatoxin-degrading activity.

Furthermore, as the composition for degradation of fungal toxin of the present invention is characterized in that it can stably exhibit the activity of degrading fungal toxin even at the temperature of 20 to 120° C., it can be applied in a broad temperature range, and thus has high usefulness.

Furthermore, the composition for degradation of fungal toxin of the present invention is characterized in that the activity of degrading fungal toxin remains stable at pH of from 6.5 to 8.5 and high temperatures like 100° C. or higher. The high temperatures like 100° C. or higher can be a temperature of 100 to 120° C., but not limited thereto.

Furthermore, according to the result of examining the activity of degrading the reference material AFB1 (aflatoxin B1), the composition for degradation of fungal toxin of the present invention showed the degradation activity of 90% for 1.5 ppm AFB1 for 24 hours at 50° C. and the degradation activity of 99% for 100 ppm AFB1 for 60 minutes at 100° C.

With regard to the composition for degradation of fungal toxin according to one embodiment of the present invention, the Aspergillus culture filtrate can be a sterile cell-free culture liquid which has been obtained by inoculating Aspergillus oryzae condinia at final concentration of 1×10⁴ to 1×10⁶ conidia/ml to 90 to 110 ml culture medium, carrying out culture for 8 to 10 days under stirring at 30±2° C., removing mycelis from the culture liquid, and filtering the resultant using a filter unit for sterilization, but it is not limited thereto.

The present invention further provides a method for degradation of fungal toxin including contacting a subject for degradation with the composition for degradation of fungi toxin of the present invention.

With regard to the method for degradation of the present invention, the fungal toxin can be preferably aflatoxin, but not limited thereto.

Furthermore, the subject for degradation can be either a food product or an animal feed, but not limited thereto.

The present invention further provides a method for production of Aspergillus culture filtrate having an activity of degrading fungal toxin including: inoculating Aspergillus conidia to a culture medium followed by culturing; and filtering a culture liquid of the Aspergillus, and it also provides an Aspergillus culture filtrate having an activity of degrading fungal toxin that is produced by the aforementioned method.

With regard to the method for production of Aspergillus culture filtrate having an activity of degrading fungal toxin according to the present invention, the Aspergillus can be Aspergillus oryzae (A. oryzae), Aspergillus terreus (A. terreus), Aspergillus sojae (A. sojae), Aspergillus nidulans (A. nidulans), Aspergillus fumigatus (A. fumigatus), or Aspergillus flavus (A. flavus), but not limited thereto.

Furthermore, the conidia can be inoculated to a culture medium at a concentration of from 1×10⁴ to 1×10⁶ conidia/ml, and preferably can be inoculated to a culture medium at a concentration of 1×10⁵ conidia/ml, and it can be cultured for 8 to 10 days under stirring at 30±2° C., but it is not limited thereto.

Furthermore, the culture medium may consist of glucose, nitrate and trace elements, and the trace elements can be iron or zinc, but not limited thereto. It may additionally comprise an element like manganese, cobalt, and copper.

The present invention further provides a food additive comprising the Aspergillus culture filtrate. When the Aspergillus culture filtrate of the present invention is used as a food additive, the Aspergillus culture filtrate may be either directly added or used in combination with other food ingredients, and it can be suitably used according to a common method. The blending amount of the effective component can be suitably set depending on the purpose of use thereof. In general, the Aspergillus culture filtrate of the present invention is added in an amount of 15 parts by weight or less, and preferably 10 parts by weight or less relative to raw materials. However, in case of the intake for a long period of time, the use amount can be lower than the described range, and, as there is no problem in terms of the safety, the effective component may be used in an amount that it higher than the described range.

Type of the food product is not particularly limited. Examples of a food product to which the aforementioned material may be added include meat products, sausages, bread, chocolate, candies, snacks, cookies, pizza, ramen, other noodles, gums, dairy products including ice cream, various soups, beverages, tea, drinks, alcohol beverages and vitamin complexes, and all food products in general sense are included therein.

The present invention still further provides an animal feed additive comprising the Aspergillus culture filtrate.

As the Aspergillus culture filtrate according to the present invention has an excellent property of degrading aflatoxin as fungi toxin, it allows good health state and improved bodyweight gain amount of livestock, and thus it can be advantageously used as an effective component of an animal feed additive.

The animal feed additive of the present invention and an animal feed comprising the same may be used with, as an auxiliary component, a material like amino acids, inorganic salts, vitamins, antibiotics, antimicrobial substances, antioxidizing, antimold enzymes, agents for improving digestion and absorption, growth promoting agents, or agents for preventing diseases.

The animal feed additive may be administered to an animal either singly or in combination with other animal feed additives in edible carrier. Furthermore, the animal feed additive can be applied as a top dressing or directly blended in an animal feed. Alternatively, separate from an animal feed, it can be easily administered, in the form of separate oral formulation, by injection or transdermal administration in combination with other components. In general, single daily dosage or divided daily dosage may be taken as it is well known in the pertinent art. When the animal feed additive is administered separately from an animal feed, the administration form of an extract can be prepared, according to combination with non-toxic pharmaceutically acceptable edible carrier, in an immediate-release formulation or a delayed-release formulation, as it is well known in the pertinent art. Examples of the edible carrier include solids and liquids such as corn starch, lactose, sucrose, bean flake, peanut oil, olive oil, sesame oil, or propylene glycol. In case of using a solid carrier, the administration form of an extract can be a tablet, a capsule, a powder, a troche, or a sugar-containing tablet, or top dressing in non-dispersion form. In case of using a liquid carrier, it may have administration form like soft gelatin capsule, syrup, liquid suspension, emulsion, or solution. Furthermore, the administration form may also include an aid such as preservative, stabilizer, wetting agent, emulsifier, or dissolution promoter.

The term “D-Tox” used herein means a composition showing excellent activity of degrading aflatoxin in which the composition is a cell-free culture filtrate of Aspergillus strain grown in food-grade medium containing human-safe edible chemicals (glucose, nitrates, minerals, cofactors, and the like). Characteristics of D-Tox according to the present invention are as described in the following Table 1.

TABLE 1
Characteristics of D-Tox

| Specifications | D-Tox | Other technologies |
|---|---|---|
| Percentage of reduction % | 90-99% | Up to 70-85% |
| Heat and processing stability | Stable | Not stable |
| Aflatoxin degradation ability | Up to 100 ppm | 0.1~5 ppm |
| Protein/non-protein based | Non-protein based | Protein based |
| Single/multiple usability | Multiple | Single |
| Time required for degradation/removal | Short e.g., 20 min | Long e.g., days |
| Types of AF detoxification reactions | Irreversible, destructed | Reversible, or binding |
| Manufacturing scale-up | Simple, cost-effective | Not easy, expensive |
| Recyclable/Environmentally friendly product | Yes | No |

Hereinbelow, the present invention is explained in greater detail in view of the Examples. However, it is evident that the following Examples are given only for exemplification of the present invention and by no means the present invention is limited to the following Examples.

### Examples

### Materials and Methods

1. Culture of Aspergillus Strains

Various Aspergillus oryzae species have been used for determining their ability to produce D-Tox (i.e., cell-free culture fermentate with aflatoxin-degrading activity), and all the strains were cultured and maintained on potato dextrose agar (PDA) medium (containing 4 g potato starch, 20 g glucose, and 15 g agar in 1 L of distilled water) at 4° C. To prepare inoculum, Aspergillus were grown on PDA for 7 days at 30±2° C. After that, asexual spores (conidia) were harvested from the PDA medium by using sterile 0.1% Tween-80 solution. The conidia were counted by using a hemocytometer and they were adjusted to 1×10⁸ conidia/ml with sterile distilled water. Fungal spore suspensions were stored at 4° C. and used within 2 weeks after the preparation.

2. Composition of Medium for Producing D-Tox

For preparing the full strength culture medium, 10.0 g D-glucose, 50 ml sodium nitrate solution, and 1.0 ml solution of trace elements were mixed and dissolved in 600 ml distilled water. After adjusting to the final volume of 1,000 ml, stirring was carried out at least for 20 minutes, and then pH was adjusted to pH 6.5 using sodium chloride. Then, according to sterilization under high pressure (50 psi for 20 minutes at 121° C.), the full strength culture medium was prepared. The sodium nitrate solution and solution of trace elements that are used for preparing the medium were prepared as described in the following Table 2.

TABLE 2
Composition of nitrate salt solution and trace element solution
Nitrate salt solution (dissolved in 1.0 liter of distilled water)

| | | |
|---|---|---|
| NaNO₃ | 120.0 | g |
| MgSO₄•7H₂O | 10.4 | g |
| KCl | 10.4 | g |
| KH₂PO₄ | 30.4 | g |

Trace element solution (dissolved in 1.0 liter of distilled water)

| | | |
|---|---|---|
| ZnSO₄•7H₂O | 22.0 | g |
| H₃BO₃ | 11.0 | g |
| MnCl₂•4H₂O | 5.0 | g |
| FeSO₄•7H₂O | 5.0 | g |
| CoCl₂•5H₂O | 1.6 | g |
| CuSO₄•5H₂O | 1.6 | g |
| (NH₄)₆Mo₇O₂₄•4H₂O | 1.1 | g |

Furthermore, composition of a culture medium for producing D-Tox in which compositions of glucose, sodium nitrate, and trace elements are changed, and type of D-Tox according to those compositions are as described in the following Table 3.

TABLE 3
Type and composition of D-Tox

| D-Tox type | Culture media composition | Notes |
|---|---|---|
| D-Tox A | 10.0 g D-glucose, | Full components as |
| | 50.0 ml nitrate salt solution, | described above Table 2 |
| | 1.0 ml trace element solution. | |
| D-Tox A_{1/2} | 5.0 g D-glucose, | Half component |
| | 25.0 ml nitrate salt solution, | of D-Tox A |
| | 0.5 ml trace element solution. | |
| D-Tox B | 10.0 g D-glucose, | Trace elements solution: |
| | 50.0 ml nitrate salt solution, | 22.0 g of ZnSO₄•7H₂O |
| | 1.0 ml trace element solution* | (zinc sulfate) and |
| | | 5.0 g of FeSO₄•7H₂O |
| | | (ferrous sulfate), dissolved |
| | | in 1.0 liter of |
| | | distilled H₂O. |
| D-Tox B_{1/2} | 5.0 g D-glucose, | Trace elements solution: |
| | 25.0 ml nitrate salt solution, | 22.0 g of ZnSO₄•7H₂O |
| | 0.5 ml trace element solution* | (zinc sulfate) and |
| | | 5.0 g of FeSO₄•7H₂O |
| | | (ferrous sulfate), dissolved |
| | | in 1.0 liter of |
| | | distilled H₂O. |
| Control | Relevant D-Tox culture | |
| | medium treated similarly, | |
| | without fungal inoculation | |

3. Preparation of D-Tox

Aspergillus oryzae conidia were inoculated into a culture medium (100 ml) to have a final concentration of 10⁵ conidia/ml and incubated for 9 days at 30±2° C. with shaking at 220 rpm. The mycelia were separated from the culture broth by filtration with four layers of Miracloth (MilliporeSigma) and the sterile cell-free culture fermentate (D-Tox) was obtained by filtering through 0.2 μm PES filter unit (Thermo Scientific, USA). D-Tox was kept such that it is stored at 4° C.

4. Preparation of Aflatoxin

A powder of AFB1 (aflatoxin B1) was purchased from Sigma Chemical Co. (St. Louis, Mo., USA). Standard solutions of AFB1 were prepared in acetonitrile at a final concentration of 10 μg/ml according to the AOAC (Association of Official Analytical Chemists) method. Thus-prepared solutions were stored at −20° C. in amber glass vials.

5. Degradation of Aflatoxin B1 (AFB1) by D-Tox

AFB1 (100 ppb, 500 ppb, 1,000 ppb, 5,000 ppb, or 100,000 ppb) was prepared and added with D-Tox. Then, degradation level of AFB1 was analyzed after the reaction under various temperature and time conditions. All the test group and control group were tested in a triplicate manner, and the degradation level of AFB1 was evaluated based on HPLC (high-performance liquid chromatography) analysis. AFB1 peak area was recorded by using ChemStation software (Agilent, USA).

TABLE 4
HPLC condition

| | |
|---|---|
| Equipment | Agilent 1100 HPLC system |
| | (degasser, autosampler, quaternary pump, coupled with a |
| | diode array detector, fluorescence detector) |
| Column | Zorbax Eclipse XDB-C18 4.6 mm × 150 mm, 3.5 μm. |
| Detection | 365 nm for UV detection, |
| wavelength | 365 nm excitation and 450 nm emission |
| | for FLD detection |
| Mobile phase | H₂O:CH₃OH:CH₃CN (50:40:10) |
| Flow rate | 0.8 ml/min |

6. Extraction of Aflatoxin

AFB1 was extracted from the sample by liquid-liquid extraction. Briefly, 1 ml of the fungal fermentate was mixed with 2 ml of chloroform and vortexed for 60 sec. The resulting mixture was then centrifuged for 5 min at 5000×g. 2 ml of the lower layer was transferred to a new glass vial. The chloroform extracts were evaporated to complete dryness. The dried extracts were dissolved in 1 ml of mobile phase. All samples were filtered through 0.22 μm syringe filter prior to HPLC analysis.

### Example 1. Analysis of Aflatoxin-Degrading Activity of D-Tox Depending on Various Reaction Temperature and Time

The inventors analyzed the degradation level of AFB1 when AFB1 (5,000 ppb) was reacted with D-Tox A (prepared by using Aspergillus oryzae NRRL3483) for 72 hours at 25° C. or 50° C. In addition, the prolonged activity was evaluated when AFB1 (1,500 ppb) was reacted with D-Tox A for 5 days at 30° C. As a result, it was found that the AFB1-degrading activity of D-Tox is proportional to the temperature and time of reaction (FIGS. 3 and 4). It was observed that more than 90% of AFB1 was degraded after 24 hours at the reaction temperature of 50° C., and it was also confirmed that, at the reaction temperature of 25° C., 89% of AFB1 was degraded after 48 hours.

Furthermore, when D-Tox A is treated with AFB1 (100,000 ppb) followed by heating for 10 minutes, it was shown that 50% of AFB1 was degraded while 96% of AFB1 was degraded after heating for 30 minutes (FIG. 5). On the other hand, AFB1 of the control group, which has not been treated with any D-Tox, maintained a stable state without showing any degradation even under heating conditions. The inventors of the present invention have confirmed that AFB1 and degradation intermediates thereof (for example, AFD1) are completely eliminated from a food product which has been treated with D-Tox for a long period of time.

### Example 2. Analysis of Aflatoxin-Degrading Activity of D-Tox Derived from Various Strains

The aflatoxin-degrading activity was compared among various Aspergillus oryzae strains and other Aspergillus strains. As a result, as it is illustrated in FIG. 6, it was confirmed that an excellent aflatoxin-degrading effect is shown from D-Tox which has been prepared by using various Aspergillus oryzae strains or other Aspergillus strains (for example, A. terrus, A. sojae, A. nidulans, A. fumigatus, and A. flavus).

### Example 3. Analysis of Aflatoxin-Degrading Activity of D-Tox Depending on Various pH Conditions

In order to evaluate the effect of pH on the reaction of degrading aflatoxin by D-Tox, the degradation activity of D-Tox on ABF1 was analyzed by the inventors of the present invention at various pH conditions (pH 6.5, pH 7.5, and pH 8.5). As a result, it was found that, at pH 6.5 conditions, ABF1 was degraded in an amount of 22% and 47%, respectively, when the reaction was carried out for 30 minutes or 60 minutes at 100° C., and at pH 7.5 conditions, ABF1 was degraded in an amount of 60% and 83.7%, respectively, when the reaction was carried out for 30 minutes or 60 minutes at 100° C. Furthermore, at pH 8.5 conditions, ABF1 was degraded in an amount of 66% and 87%, respectively, when the reaction was carried out for 30 minutes or 60 minutes at 100° C. (FIG. 7). Based on these results, it was recognized that the aflatoxin-degrading activity of D-Tox is at least about 3 times more stable at pH 7.5 and pH 8.5 conditions than that of pH 6.5 conditions, and the aflatoxin-degrading activity is stably maintained at high reaction temperatures like 100° C.

What is claimed is:

1. A composition for degradation of fungal toxin comprising Aspergillus culture filtrate as an effective component.

2. The composition for degradation of fungal toxin according to claim 1, wherein the Aspergillus is Aspergillus oryzae, Aspergillus terreus, Aspergillus sojae, Aspergillus nidulans, Aspergillus fumigatus, or Aspergillus flavus.

3. The composition for degradation of fungal toxin according to claim 1, wherein the fungi toxin is aflatoxin.

4. The composition for degradation of fungal toxin according to claim 1, wherein the composition exhibits the activity of degrading fungal toxin at a temperature of 20 to 120° C.

5. The composition for degradation of fungal toxin according to claim 1, wherein the composition has stable activity of degrading fungal toxin at pH of from 6.5 to 8.5 and high temperatures of 100° C. or higher.

6. A method for degradation of fungal toxin including contacting a subject for degradation with the composition of claim 1.

7. The method according to claim 6, wherein the fungal toxin is aflatoxin.

8. The method according to claim 6, wherein the subject for degradation is a food product or an animal feed.

9. A method for production of Aspergillus culture filtrate having an activity of degrading fungal toxin including:
inoculating Aspergillus conidia to a culture medium followed by culturing; and
filtering a culture liquid of the Aspergillus.

10. The method for production of Aspergillus culture filtrate having an activity of degrading fungal toxin according to claim 9, wherein the Aspergillus is Aspergillus oryzae, Aspergillus terreus, Aspergillus sojae, Aspergillus nidulans, Aspergillus fumigatus, or Aspergillus flavus.

11. The method for production of Aspergillus culture filtrate having an activity of degrading fungal toxin according to claim 9, wherein the culture medium consists of glucose, nitrates, and trace elements.

12. The method for production of Aspergillus culture filtrate having an activity of degrading fungal toxin according to claim 9, wherein the fungal toxin is aflatoxin.

13. An Aspergillus culture filtrate having an activity of degrading fungal toxin that is produced by the method of claim 9.

14. A food additive comprising an Aspergillus culture filtrate having an activity of degrading fungal toxin.

15. An animal feed additive comprising an Aspergillus culture filtrate having an activity of degrading fungal toxin.
